(19) United States
(12) Patent Application Publication
(10) Pub. No.: US 2015/0276730 A1
(43) Pub. Date: Oct. 1, 2015

(54) PATHOGEN DETECTION BY STRAIN-SPECIFIC BACTERIAL RECOGNITION AND PLASMONIC AMPLIFICATION AND READOUT

(71) Applicant: UNIVERSITY OF NOTRE DAME DU LAC OFFICE OF TECHNOLOGY TRANSFER, Notre Dame, IN (US)

(72) Inventors: Marvin J. Miller, South Bend, IN (US); Cheng Ji, New York, NY (US); Paul Bohn, Grainger, IN (US); Sean Branagan, South Bend, IN (US); Yang Yang, South Bend, IN (US)

(73) Assignee: UNIVERSITY OF NOTRE DAME DU LAC OFFICE OF TECHNOLOGY TRANSFER, Notre Dame, IN (US)

(21) Appl. No.: 14/440,345

(22) PCT Filed: Nov. 1, 2013
(86) PCT No.: PCT/US2013/068175

§ 371 Date: May 1, 2015

Related U.S. Application Data
(60) Provisional application No. 61/796,044, filed on Nov. 1, 2012.

(51) Int. Cl. G01N 33/543; B03C 5/00; G01N 27/447; G01N 33/569; G01N 27/327

(52) U.S. Cl. CPC G01N 33/54393; G01N 33/56911; G01N 27/3275; G01N 27/44756; B03C 5/005

(57) ABSTRACT

The present invention relates to a method and kit for detecting bacteria in a sample. Substrate having a surface comprising an interdigitated Au electrode array and a plurality of siderophores specific to the bacteria and covalently attached to the surface. In one embodiment, the siderophore may contain a free OH (alcohol), amine, or carboxylic acid to which linker may be attached via ester (on the OH), amide (on the amine) or reverse the ester or amide using the siderophore carboxyl. The linker chain can then be short or long with and without heteroatom substitution to improve solubility as needed. The linker can terminate with a thiol which will react with a gold surface. Alternatively, the linker can terminate with another alcohol, amine or acid which can then be attached to corresponding functionality on the surface of choice.

The entire contents of US provisional applications 61/899,154 filed Nov. 1, 2012, and 61/894,770, filed Oct. 23, 2013 are hereby incorporated by reference.

### BRIEF DESCRIPTION OF THE SEVERAL EMBODIMENTS

An improved method of detecting pathogenic bacteria based on microbial iron chelators is disclosed. The technology uses selective recognition of siderophores to identify and characterize different types of bacteria. Combining these tasks enables the development of a rapid diagnostic test for use in health care laboratories or at the point-of-care. The technology can be adapted for single strains of bacteria or multiple bacterial analyses from the same microfluid sample. In practice, the device is realized in one of two formats: (1) a microfluidic multichannel affinity chromatography and detection system based on covalent attachment of bacteria to siderophores and analogs to the surface of separate channels in the microfluidic device; and (2) affinity-based pulldown onto a solid substrate followed by complementary recognition by gold nanoparticles and subsequent amplification by Ag particle nucleation. In format (1) passage of sub-microliter volumes of sample through the device will allow exposure to the adsorbed siderophores that specifically recognize and tightly bind the respective bacteria. The bacteria thus pulled down will be detected using one of various sensing techniques. In a primary development of the invention, label-free surface-plasmon (SPR) detection with an external reader is used. In format (2) the primary recognition event, which results in a surface bound bacterium, is followed by a second affinity recognition event using Au nanoparticles tagged with the same siderophore. Subsequently, these nanoparticles are used as nucleation sites for the growth of high optical density Ag particles by reduction of solution-phase Ag(I) via electroless deposition. Format (1) is envisioned to target hospital or public health applications, whereas format (2) is aimed at resource-limited settings, such as found in the developing world. The optimal device will be low cost, easy to use and extraordinarily sensitive. The following describes a representative application focusing on rapid diagnosis of tuberculosis to demonstrate the potential of the plan and then illustrates planned applications to detect multidrug-resistant organisms (MDROs) and/or nosocomial pathogens, particularly Acinetobacter baumannii, Pseudomonas aeruginosa and methicillin-resistant Staphylococcus aureus (MRSA).

### BACKGROUND

Iron is essential for the growth of virtually all forms of life including Mycobacterium tuberculosis (Mtb), Acinetobacter baumannii, Pseudomonas aeruginosa and methicillin-resistant Staphylococcus aureus (MRSA). Since Fe(III) is very insoluble at physiological pH, microbes have evolved exquisitely specific processes for iron sequestration that often involve active transport through an otherwise impermeable outer membrane. Bacterial iron acquisition is essential for pathogenicity, and provides an attractive and heretofore little-used target for the development of microbe-selective biomarkers for selective detection. Acquisition of iron by bacteria at the infection site depends on the presence of soluble Fe(III) complexes generated from iron sources. These solubilized Fe(III)-complexes (generically called siderophores) must then be sequestered by the bacteria to initiate iron transport across the cell envelope. Siderophores have been used in a number of applications requiring bacterial recognition due to their inherent specificity.

Physicians are in need of an improved method for identifying pathogenic bacteria, especially those drug-resistant strains which currently cause the majority of deaths within health care facilities. Examples include methicillin-resistant Staphylococcus aureus (MRSA), multidrug resistant Myobacterium tuberculosis (Mtb), Pseudomonas aeruginosa, and multidrug-resistant Acinetohacter baumannii (MDRAB). Since any delay in treatment of an infection increases the likelihood of a fatality, physicians frequently begin treatment before the exact strain is identified. This leads to sub-optimal care, where for example a broad spectrum antibiotic is prescribed where a tailored drug is necessary, or an insufficient dose is prescribed, both of which contribute to further drug resistance by the pathogen.

Current diagnostic methods generally fall into one of four categories: (1) specific culturing of the organism followed by visual inspection for identifying phenotypic characteristics, (2) detection of pathogen-specific antibodies produced by the patient, (3) immunological-based detection of specific pathogen products, e.g., exotoxins, and (4) genetic sequencing. All of these methods require a lot of time, up to several days, in order to reach an accurate diagnosis. For example, the fastest rapid-screening technique for MRSA currently available, quick multiplex immunocapture-coupled PCR (qMRSA), produces a diagnosis using as few as 5 genome copies in approximately 22 hours, versus up to 4 days using conventional culture. Even this four-fold improvement is insufficient to allow point-of-care diagnostics, which is ideal for patient care. Similarly, two of the above methods, antibody detection and immunological-based detection of infection byproducts, require an immune response by the patient after infection; patients with compromised immune systems are among those most at risk of death from MRSA and tuberculosis. Although some rapid diagnostic tests have been developed in recent years, accurate clinical diagnosis (identification & characterization) still requires confirmation by another (slow) technique. Therefore, initial treatment of a bacterial infection is typically begun without confirmation of the specific infection type, since any delay in treatment could result in a fatal infection. Some initial work on siderophore-based detection has been done, but these techniques require microscopic imaging and/or significant post-processing in order to detect a bacterial strain. Therefore, a significant need exists for an improved detection technique, based on microbial affinity, which is fast, selective, and analytically efficient.

Correct initial treatment (which requires a fast and accurate initial diagnosis) has been found to significantly improve patient outcomes, especially among drug-resistant infections acquired in hospitals. In contrast, failure to quickly recognize and treat patients with MTB leads to increased mortality, nosocomial infections, and further resistance to antimicrobial drugs. Patients with traumatic injuries are especially prone to wound colonization and infection with strains of both Gram positive and Gram negative forms of bacteria. Proper treatment requires rapid and accurate diagnosis of the infectious organism, preferably in the field with minimal delay. The diagnostic method disclosed here, in its most portable practice, is intended to have an immediate and positive impact on survival of such patients. The need to reduce the evolutionary forces driving antibiotic resistance is another utility for fast and accurate bacterial diagnosis. Mistaken prescriptions of antibiotics to treat viral infections, for example, could be reduced by the availability of a cheap and user-friendly bacterial diagnostic test. Significant economic growth within point-of-care diagnostics has already been realized, and the market is projected to approximately double within the next decade. Surface plasmon resonance (SPR), and especially second generation SPR techniques amenable to miniaturization, are expected to play a central role in chemical analysis of the future. Techniques which do not require microscopic imaging, such as phase-shift SPR, wavevector-resolved SPR, and others are the preferred technique for adapting to siderophore-mediated bacterial sensing. Finally, the technique of electroless deposition is anticipated to form the basis of a label-free test strip kit, which would not require a reader of any kind, and is thus deployable in resource-poor environments

The technology disclosed here uses the exquisitely selective recognition of siderophores (microbial iron chelators) by different types of bacteria and will be able to differentiate bacteria and allow for rapid diagnostics. The technology can be adapted for single strains of bacteria or multiple bacterial analyses from the same microfluid sample. In brief, the device will be a microfluidic multichannel affinity chromatography and detection system based on covalent attachment of bacteria specific siderophores and analogs to the surface of separate channels in the microfluidic device. Passage of microliter volumes of sample through the device will allow exposure to the adsorbed siderophores that specifically recognize and tightly bind the respective bacteria. The bacteria thus pulled down will be detected using one of various sensing techniques. In a primary development of the invention, label-free surface-plasmon (SPR) detection using an external reader will be developed (format 1). Alternatively, no reader will be required where the sensor is adapted to use electroless deposition of a metal onto a label-free test strip (format 2). The optimal device will be low-cost, easy to use and extraordinarily sensitive—down to the selective detection of a single bacteria cell. The following describes a representative application focusing on rapid diagnosis of tuberculosis to demonstrate the potential of the plan and then illustrates planned applications to detect multidrug-resistant organisms (MDROs) and/or nosocomial pathogens, particularly Acinetobacter baumannii, Pseudomonas aeruginosa and methicillin-resistant Staphylococcus aureus (MRSA).

Iron is essential for the growth of virtually all forms of life including Mtb, Acinetobacter baumannii, Pseudomonas aeruginosa and methicilin-resistant Staphylococcus aureus (MRSA). Since Fe(III) is very insoluble at physiological pH, microbes have evolved very specific processes for iron sequestration that often involve active transport through an otherwise impermeable outer membrane. Bacterial iron acquisition is essential for pathogenicity, and provides an attractive and heretofore little-used target for the development of microbe-selective antibiotics and biomarkers for selective detection. Acquisition of iron by bacteria at the infection site depends on the presence of soluble Fe(III) complexes generated from iron sources. These solubilized Fe(III)-complexes (generically called siderophores) must then be sequestered by the bacteria to initiate iron transport across the cell envelope. For example, the unequivocal importance of the specific siderophore mycobactin T to the growth of Mtb has been established by showing that a mutant of Mtb lacking a gene from mycobactin biosynthesis had a considerably decreased ability to grow in human macrophages. The Miller group has synthesized mycobactin T (1), the Mtb specific siderophore, analogs and, most recently, a conjugate (3) with artemisinin Although the antimalarial agent, artemisinin (2) itself is not active against tuberculosis, conjugation to a Mtb specific siderophore (microbial iron chelator) analog induces significant and selective anti-tuberculosis activity, including activity against MDR and XDR strains of Mtb. Physicochemical and whole cell studies indicate that ferric to ferrous reduction of the iron complex of the conjugate initiates the expected bactericidal Fenton-type radical chemistry on the artemisinin component. Thus, this “Trojan Horse” approach demonstrates that new pathogen selective therapeutic agents can be generated in which the iron component of the delivery vehicle also participates in triggering the antibiotic activity. The result is that the critical iron uptake machinery of Mtb is demonstrably selective and thus is uniquely suited for design of a sensitive, selective and non-invasive diagnostic tool. As a further indication of its microbe selectivity, we found that conjugate 3 was not active against a broad set of Gram-positive and Gram-negative bacteria at the highest levels tested (2 mM). As still another indication of the unique anti-Mtb selectivity of 3, it was tested and found to have negligible activity (>100 fold less) against a number of fast growing strains of mycobacteria (M. vaccae, M. smegmatis, M. aurum and M. fortuitum). Thus, the antibiotic activity of conjugate 3 is microbe-selective, because of exploitation of the unique and essential iron assimilation process, as anticipated. The Miller group also previously reported the design, syntheses and antimicrobial activity of unnatural carbacephalosporin siderophore conjugates 4-5 with separate hydroxamic acid-based and catechol-based siderophore components. As expected, detailed biological assays revealed that the hydroxamate- and catechol-containing conjugates utilized different outer membrane receptor proteins to initiate cellular entry (Fhu and cir, respectively) and exquisite bacteria selectivity, including remarkable activity against pathogens that cause serious health risks to military personnel.

Pseudomonas aeruginosa produces very specific siderophores, including pyoverdine (6, R=OH) and related studies indicate the potential for use as pseudomonally selective affinity agents. We have optimized fermentation processes to obtain natural pyoverdine free acid (R=OH) that is directly suitable for coupling to pegylated thiols needed for the selective detection methodology described below.

As described above, the diagnostic method of the invention targets a fundamental metabolic activity of specific bacteria, the siderophore-mediated metabolic uptake of iron, to mediate the capture and confinement of targeted pathogens. In both formats (1) and (2) the bacteria-specific siderophore (e.g., the siderophore component of 3-6) is anchored to a surface (gold or polymer) in such a way that the targeted bacteria, while attempting to ingest the siderophore, also become anchored to the surface—a process that will be sensitively detected using label-free SPR detection. For example, FIG. 3 illustrates particular realization of format (1) in which SPR imaging is used to distinguish between microfluidic channels that contain only the capture agent and those in which an analyte has been captured (sample). The siderophore-bioconjugates are functionalized to the capture surface (pegylated Au, chosen for resistance to non-specific adsorption) via a heterobifunctional linker, allowing us to simultaneously mitigate against non-specific adsorption, present competent capture motifs well-separated from the underlying protective layer and capture bacteria with both exquisite sensitivity and selectivity.

Furthermore, the potential high-cost driver derived from the use of Au can be circumvented either by constructing a demountable SPR platform in which the sampling is implemented with a “throw-away” plastic element that has the microfluidic channels embossed into it or by exploiting the localized surface plasmon effect with inexpensive Au colloid active layers. After collecting the sample directly on the disposable element, it is mated directly onto the field-deployable reader. The reader—essentially a miniaturized cabinet with light source, coupling optics, detector and readout electronics—is ruggedized so that it can be maintained by a semi-skilled person on a location-by-location basis.

As shown in FIG. 3, the format (1) detection platform combines (a) self-referencing microfluidic multi-lane arrays; (b) SPR imaging/angle shifts for readout; and (c) reusable fluidic chips. Furthermore, carrying out the recognition event in a microfluidic format accrues inherent mass transport advantages meaning that measurements can be cycled faster than with benchscale flow cells. In addition to the specificity provided by the siderophore, the plasmonic readout easily has the sensitivity to detect a single pathogen organism in the active area (typically 50 μm (micrometers) wide by 1 mm long). The ultimate solution-referenced limit of detection (LOD) is determined by the capture efficiency, and LODs of a few units mL⁻¹ are readily attainable. We are currently optimizing the surface derivatization chemistries used to anchor the siderophores for optimal bacterial recognition and capture.

Strong motivation exists for a pathogen diagnostic test which requires no reader at all, and is usable in the field by personnel with no training, a paradigm known as point-of-care diagnostics. Format (2) embodies an alternative practice of the invention. As shown in FIG. 4, a test substrate is functionalized with an artificial siderophore, which is selective for the targeted pathogen. A bacterial cell is captured on the surface, similar to that described above. In a second step, the remaining species in solution are rinsed away in a buffer solution. In 4(C), a solution of functionalized metallic nanoparticles is introduced, which binds to the surface of the bacteria. The molecular recognition moiety in (C) may be a siderophore, an antibody, or some other species which binds to the bacteria present on the surface. Since the selection (identification) of the bacteria has already taken place by the immobilized siderophore in 4(A), the subsequent advantage of the nucleating metallic nanoparticles need not be species- or strain-selective, a distinct advantage in ease of use compared to format (1). The final step of the diagnostic test, the development step, involves a solution of metal ions (Ag for example) and an organic reductant. Such a solution is well-known to result in a thick film of metal wherever a nucleation site exists. Thus, the test strip described here is label-free, does not require a reader, and maintains the benefits of siderophore-mediated sensing described above.

### Linkers

The linker is not particularly limited, so long as it can attach the siderophore to the surface and not interfere or substantially interfere with the binding ability of the siderophore to the bacteria. In one embodiment, the siderophore may contain a free OH (alcohol), amine, or carboxylic acid to which linker may be attached via ester (on the OH), amide (on the amine) or reverse the ester or amide using the siderophore carboxyl. The linker chain can then be short or long with and without heteroatom substitution to improve solubility as needed. The linker can terminate with a thiol which will react with a gold surface. Alternatively, the linker can terminate with another alcohol, amine or acid which can then be attached to corresponding functionality on the surface of choice. Some examples of suitable linkers for bioconjugation may be found in Bioconjugate Techniques by Greg T. Heranson, Academic Press, 1996, incorporated herein by reference.

Other examples of linkers are given below:

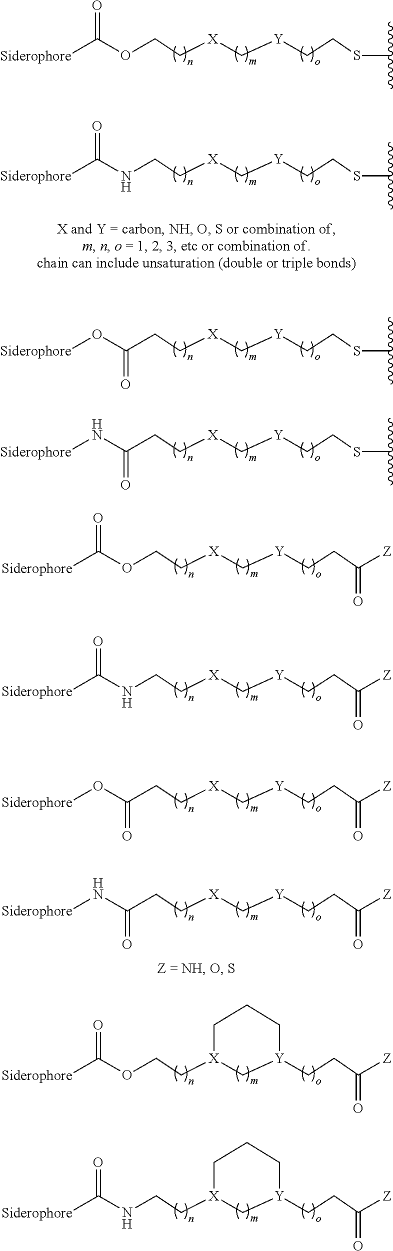

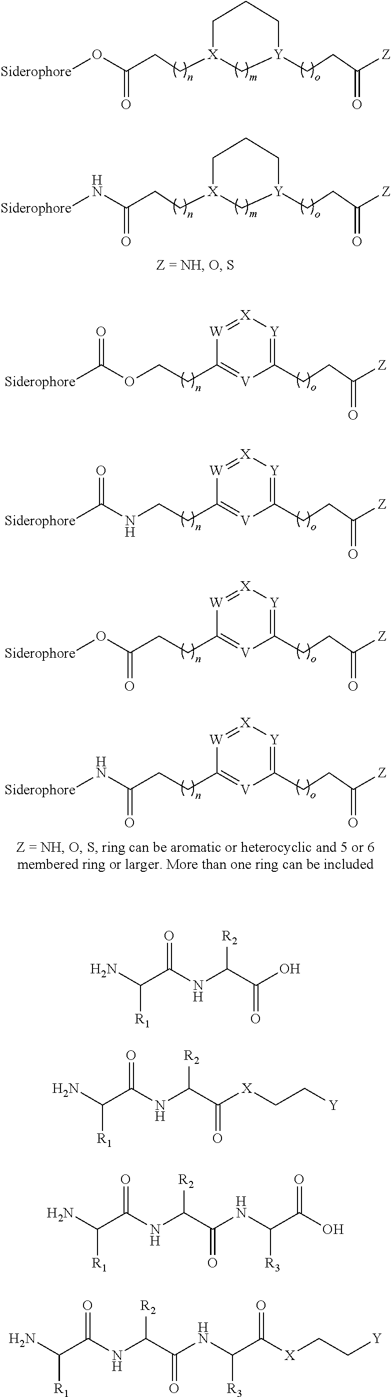

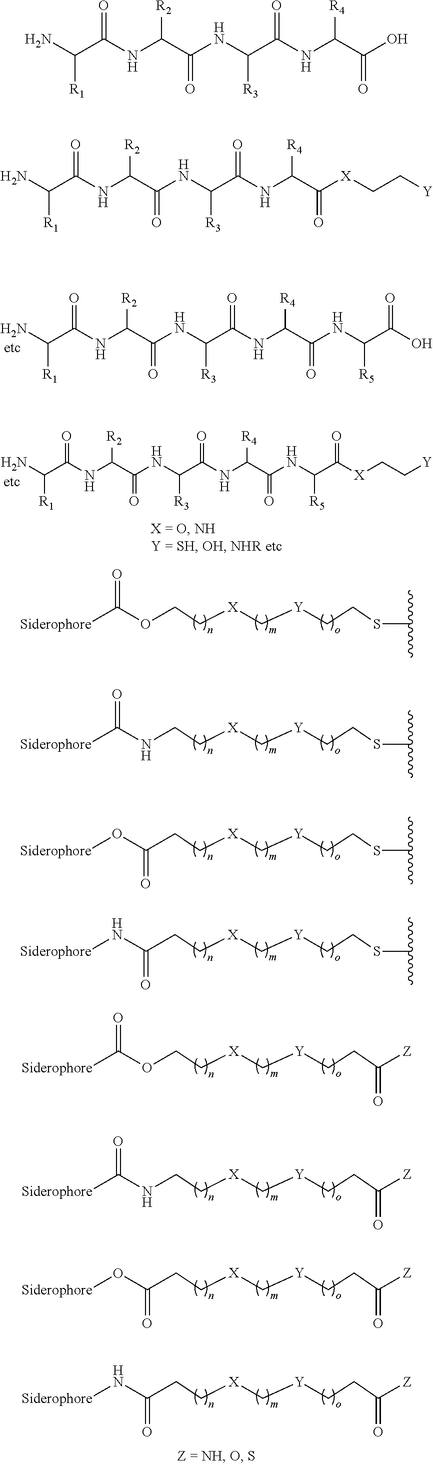

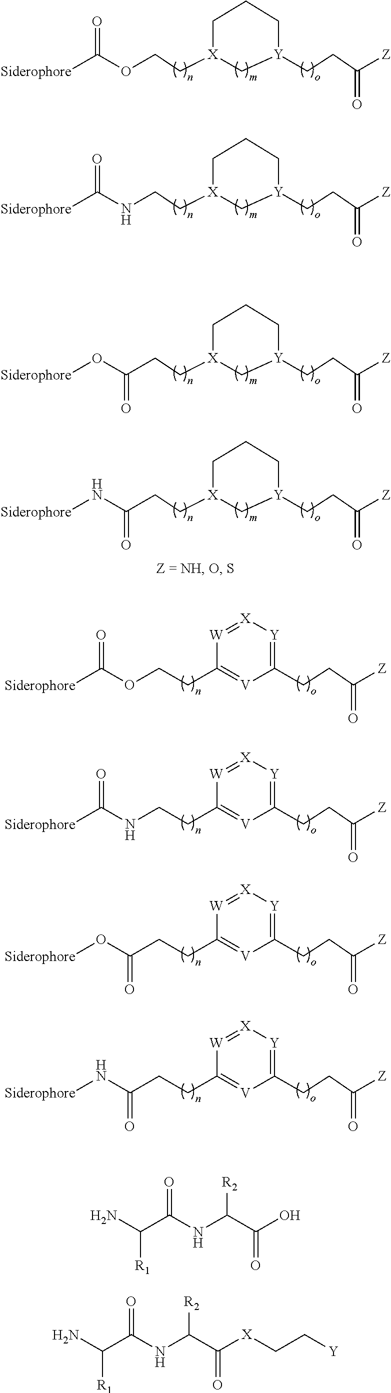

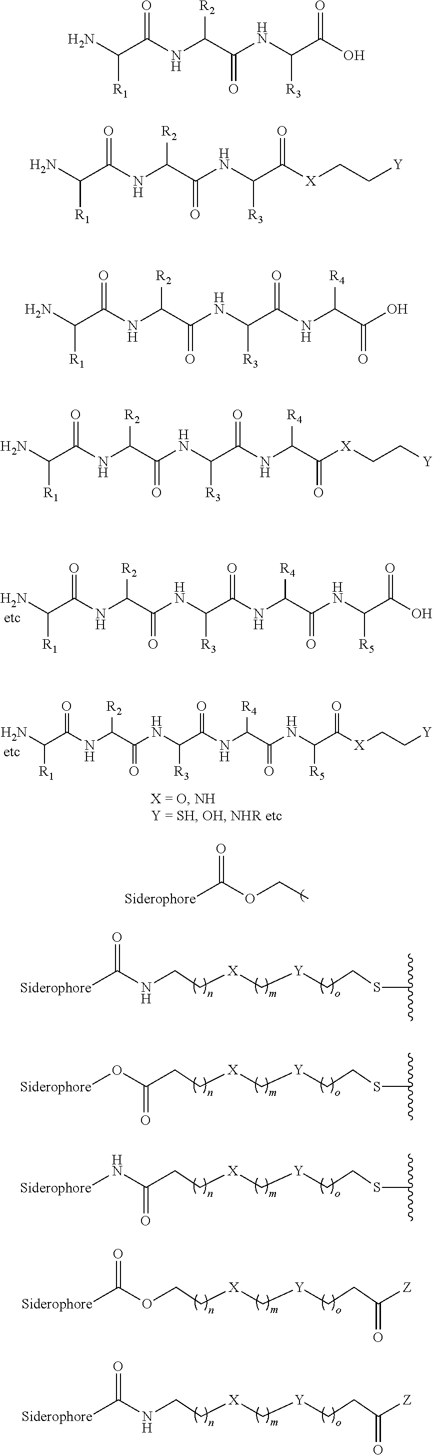

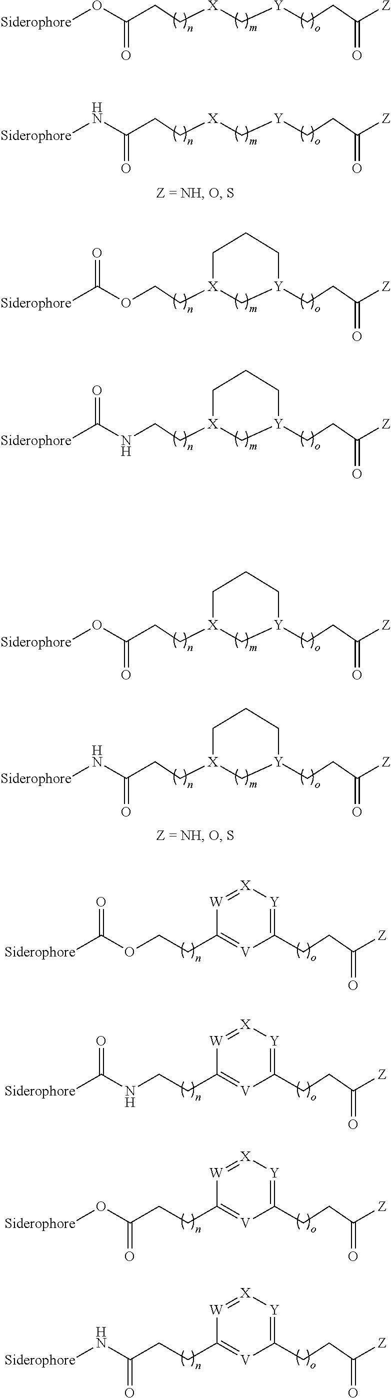

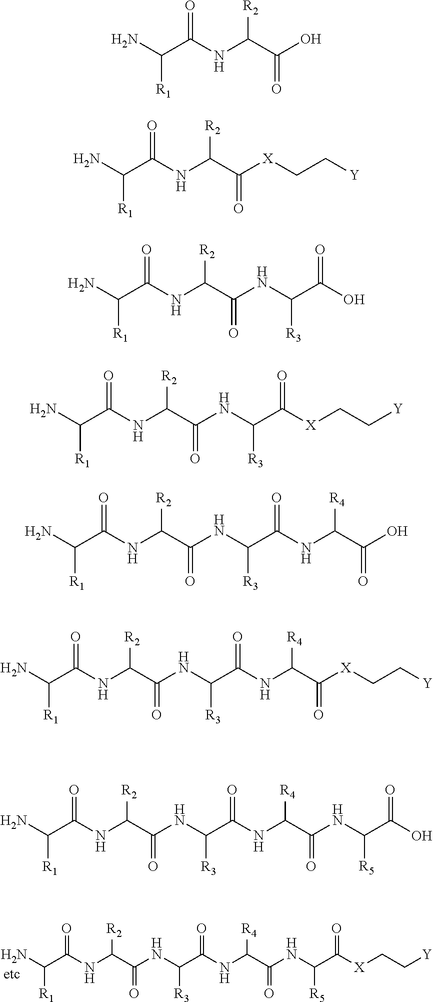

Natural Siderophore

Desferrioxamine A1
Desferrioxamine A2
Desferrioxamine B
Desferrioxamine D1
Desferrioxamine D2
Desferrioxamine E
Desferrioxamine G1
Desferrioxamine G2A
Desferrioxamine G2B
Desferrioxamine G2C
Desferrioxamine H
Desferrioxamine T1
Desferrioxamine T2
Desferrioxamine T3
Desferrioxamine T7
Desferrioxamine T8
Desferrioxamine X1
Desferrioxamine X2
Desferrioxamine X3
Desferrioxamine X4
Desferrioxamine Et1
Desferrioxamine Et2
Desferrioxamine Et3
Desferrioxamine Te1
Desferrioxamine Te2
Desferrioxamine Te3
Desferrioxamine P1
Ferrichrome
Ferrichrome C
Ferricrocin
Sake Colorant A
Ferrichrysin
Ferrichrome A
Ferrirubin
Ferrirhodin
Malonichrome
Asperchrome A
Asperchrome B1
Asperchrome B2
Asperchrome B3
Asperchrome C
Asperchrome D1
Asperchrome D2
Asperchrome D3
Asperchrome E
Asperchrome F1
Asperchrome F2
Asperchrome F3
Tetraglycine ferrichrome
Des(diserylglycyl)-ferrirhodin
Basidiochrome
Triacetylfusarinine
Fusarinine C
Fusarinine B
Neurosporin
Coprogen
Coprogen B (Desacetylcoprogen)
Triornicin (Isoneocoprogen I)
Isotriornicin (Neocoprogen I)
Neocoprogen II
Dimethylcoprogen
Dimethylneocoprogen I
Dimethyltriornicin
Hydroxycopropen
Hydroxy-neocoprogen I
Hydroxyisoneocoprogen I
Palmitoylcoprogen
Amphibactin B
Amphibactin C
Amphibactin D
Amphibactin E
Amphibactin F
Amphibactin G
Amphibactin H
Amphibactin I
Ferrocin A
Coelichelin
Exochelin MS
Vicibactin
Enterobactin (Enterochelin)
Agrobactin
Parabactin
Fluvibactin
Agrobactin A
Parabactin A
Vibriobactin
Vulnibactin
Protochelin
Corynebactin
Bacillibactin
Salmochelin S4
Salmochelin S2
Rhizoferrin
Rhizoferrin analogues
Enantio Rhizoferrin
Staphyloferrin A
Vibrioferrin
Achromobactin
Mycobactin P
Mycobactin A
Mycobactin F
Mycobactin H
Mycobactin M
Mycobactin N
Mycobactin R
Mycobactin S
Mycobactin T
Mycobactin Av
Mycobactin NA (Nocobactin)
Mycobactin J
Formobactin
Nocobactin NA
Carboxymycobactin
Carboxymycobactin 1
Carboxymycobactin 2
Carboxymycobactin 3
Carboxymycobactin 4
Pyoverdin 6.1 (Pseudobactin)
Pyoverdin 6.2
Pyoverdin 6.3 (Pyoverdin Thai)
Pyoverdin 6.4 (Pyoverdin 9AW)
Pyoverdin 6.5
Pyoverdin 6.6
Isopyoverdin 6.7, (Isopyoverdin BTP1)
Pyoverdin 6.8
Pyoverdin 7.1
Pyoverdin 7.2, (Pyoverdin BTP2)
Pyoverdin 7.3, (Pyoverdin G + R)
Pyoverdin 7.4, (Pyoverdin PVD)
Pyoverdin 7.5, (Pyoverdin TII)
Pyoverdin 7.6
Pyoverdin 7.7,
Pyoverdin 7.8, (Pyoverdin PL8)
, Pyoverdin 7.9, (Pyoverdin 11370)
Pyoverdin
Pyoverdin 7.11, (Pyoverdin 19310)
Pyoverdin 7.12, (Pyoverdin 13525)
Isopyoverdin 7.13, (Isopyoverdin 90-33)
Pyoverdin 7.14, (Pyoverdin R′)
Pyoverdin 7.15
Pyoverdin 7.16, (Pyoverdin 96-312)
Pyoverdin 7.17
Pyoverdin 7.18
Pyoverdin 7.19
Pyoverdin 8.1, (Pyoverdin A214)
Pyoverdin 8.2, (Pyoverdin P19)
Pyoverdin 8.3, (Pyoverdin D-TR133)
Pyoverdin 8.4, (Pyoverdin 90-51)
Pyoverdin 8.5
Pyoverdin 8.6, (Pyoverdin 96-318)
Pyoverdin 8.7, (Pyoverdin I-III)
Pyoverdin 8.8, (Pyoverdin CHAO)
Pyoverdin 8.9, (Pyoverdin E)
Pyoverdin 9.1
Pyoverdin 9.2, (Pyoverdin Pau)
Pyoverdin 9.3
Pyoverdin 9.4
Pyoverdin 9.5, (Pyoverdin 2392)
Pyoverdin 9.6
Pyoverdin 9.7, (Pseudobactin 589A)
Pyoverdin 9.8, (Pyoverdin 2461)
Pyoverdin 9.9
Pyoverdin 9.10, (Pyoverdin 95-275)
Pyoverdin 9.11, (Pyoverdin C)
Pyoverdin 9.12
Pyoverdin 10.1, (Pyoverdin 2798)
Pyoverdin 10.2
Pyoverdin 10.3, (Pyoverdin 17400)
Pyoverdin 10.4
Pyoverdin 10.5, (Pyoverdin 18-1)
Pyoverdin 10.6, (Pyoverdin 1, 2)
Isopyoverdin 10.7, (Isopyoverdin 90-44)
Pyoverdin 10.8
Pyoverdin 10.9, (Pyoverdin 2192)
Pyoverdin 10.10
Pyoverdin 11.1, (Pyoverdin 51W)
Pyoverdin 11.2, (pyoverdin 12)
Pyoverdin 12.1, (Pyoverdin GM)
Pyoverdin 12.2, (Pyoverdin 1547)
Azoverdin
Azotobactin 87
Azotobactin D
Heterobactin A
Ornibactin - C4
Ornibactin - C6
Ornibactin - C8
Aquachelin A
Aquachelin B
Aquachelin C
Aquachelin D
Marinobactin A
Marinobactin B
Marinobactin C
Marinobactin D1
Marinobactin D2
Marinobactin E
Loihichelin A
Loihichelin B
Loihichelin C
Loihichelin D
Loihichelin E
Loihichelin F
Schizokinen
Aerobactin
Arthrobactin
Rhizobactin 1021
Nannochelin A
Nannochelin B
Nannochelin C
Acinetoferrin
Ochrobactin A
Ochrobactin B
Ochrobactin C
Snychobactin A
Snychobactin B
nychobactin C
Mugineic acid
3-Hydroxymugineic acid
2′-Deoxymugineic acid
Avenic acid
Distichonic acid
Deoxydistichonic acid
Rhizobactin
Staphyloferrin B
Alterobactin A
Alterobactin B
Pseudoalterobactin A
Pseudoalterobactin B
Petrobactin
Petrobactin sulphonate
Petrobactin disulphonate
Fusarinine A
Exochelin MN
Ornicorrugatin
Maduraferrin
Alcaligin
Putrebactin
Bisucaberin
Rhodotrulic acid
Dimerum acid
Amycolachrome
Azotochelin, (Azotobactin),
Myxochelin
Amonabactin T789
Amonabactin P750
Amonabactin T732
Amonabactin P693
Salmochelin S1
Serratiochelin
Anachelin 1
Anachelin 2
Pistillarin
Anguibactin
Acinetobactin
Yersiniabactin
Micacocidin
Deoxyschizokinen
Heterobactin B
Desferrithiocin
Pyochelin
Thiazostatin
Enantio-Pyochelin
2,3-Dihydroxybenzoylserine
Salmochelin SX
Citrate
Chrysobactin
Aminochelin
Siderochelin A
Aspergillic acid
Itoic acid
Cepabactin
Pyridoxatin
Quinolobactin
Ferrimycin A
Salmycin A
Albomycin

1. A device for detecting bacteria in a sample, comprising:
a substrate having a surface comprising an interdigitated Au electrode array; and
a plurality of siderophores specific to the bacteria and covalently attached to the surface.

2. The device of claim 1, wherein the surface further comprises polymer, silica, or a combination thereof.

3. The device of claim 1, wherein the siderophores are attached directly or indirectly through a linking group.

4. The device of claim 1, wherein the siderophore is a naturally occurring or synthetic siderophore.

5. A diagnostic test strip for detecting bacteria in a sample, comprising:
a substrate having a surface other than gold or glass; and
a plurality of siderophores specific to the bacteria and covalently attached to the surface.

6. The test strip of claim 5, wherein the substrate surface is paper, polymer, silica, quartz, or combination thereof.

7. The test strip of claim 5, wherein the siderophores are attached directly or indirectly through a linking group.

8. The test strip of claim 5, wherein the siderophore is naturally occurring or synthetic.

9. A method for detecting bacteria in a sample, comprising:
contacting the sample with a substrate having a surface comprising an interdigitated Au electrode array (IDE) and a plurality of siderophores specific to the bacteria and covalently attached to the surface;
dielectrophoresing the sample over the IDE, to effect a binding of the bacteria, if present in the sample, to one or more of the siderophores;
detecting the presence or absence of the bacteria so bound using Surface Plasmon Resonance.

10. The method of claim 9, wherein the surface further comprises glass, polymer, silica, or a combination thereof.

11. The method of claim 9, wherein the siderophores are attached directly or indirectly through a linking group.

12. The method of claim 9, wherein the siderophore is a naturally occurring or synthetic siderophore.

13. The method of claim 9, wherein the bacteria is present in the sample and is detected.

14. The method of claim 9, wherein the bacteria is not present in the sample and is not detected.

15. The method of claim 9, wherein the sample comprises a mixture of bacteria for which the siderophore is specific and bacteria for which the siderophore is not specific, and wherein the bacteria for which the siderophore is specific is detected and bacteria for which the siderophore is not specific is not detected.

16. The method of claim 9, further comprising quantifying the detected bacteria.

17. The method of claim 9, further comprising one or more washing steps between one or more of the contacting, dielectrophoresing, and detecting.

18. A method for detecting bacteria in a sample, comprising:
contacting the sample with a substrate having a surface comprising a plurality of covalently attached siderophores specific to the bacteria, to effect a binding of one or more of the bacteria, if present in the sample, to one or more of the siderophores;
detecting the presence or absence of the bacteria so bound using Surface Plasmon Resonance.

19. The method of claim 18, wherein the surface further comprises glass, polymer, silica, or a combination thereof.

20. The method of claim 18, wherein the siderophores are attached directly or indirectly through a linking group.

21-56. (canceled)
